(19) United States
(12) Patent Application Publication
(10) Pub. No.: US 2014/0169519 A1
(43) Pub. Date: Jun. 19, 2014

(54) Cone-beam CT Scanning

(71) Applicant: ELEKTA AB (PUBL), Stockholm (SE)

(72) Inventors: Per Harald Bergfjord, Middlesex (GB); Duncan Neil Bourne, Surrey (GB); Stephen Young, West Sussex (GB)

(73) Assignee: ELEKTA AB (PUBL), Stockholm (SE)

(21) Appl. No.: 13/717,792

(22) Filed: Dec. 18, 2012

(51) Int. Cl. A61B 6/00; A61B 6/03

(52) U.S. Cl. CPC A61B 6/482; A61B 6/035
USPC 378/5; 378/16

(57) ABSTRACT

Combined systems that rely on a single source able to switch between therapeutic emissions and diagnostic emissions for a cone-beam CT scanner can be improved by rotating the collimator during CT scanning to allow a wider maximum aperture. The detector can also be positioned in an offset manner so as to take best advantage of this aperture. The rotated position for a collimator with a rectangular aperture (such as a square) can be one in which a diagonal of the aperture lies transverse to the plane swept out by the beam axis during rotation of the mount. More generally, where the aperture has at least one straight edge, the predetermined position is one in which the straight edge lies at an oblique angle to the plane swept out by the beam axis during rotation of the mount.

### FIELD OF THE INVENTION

The present invention relates to cone-beam CT scanning.

### BACKGROUND ART

Computed tomography techniques were first suggested in the 1960s, with practical implementation beginning in the 1970s. The essential principle is that a number of projections are obtained from a number of rotational directions around a single axis of rotation, showing the x-ray attenuation after passing through the object under investigation. Computational techniques are applied to this plurality of projections, to yield a three-dimensional image of the interior of the object. Contrast in the image is derived from the different attenuation rates of the different materials making up the object, and the overall image quality is dependent on the provision of an adequate number of projections. The basic process is set out in U.S. Pat. No. 3,106,640 but has been developed considerably since then.

Typically, a CT scanner will comprise an x-ray source mounted in a rotateable manner around an axis, such as on a ring or a gantry, together with either a single detector mounted opposite the source or a plurality of detectors arranged around the ring. The scanner will be rotated around the axis and will emit pulses of radiation at a predetermined frequency, i.e. with a predetermined time period between them. These pulses will then be detected after attenuation and the resulting series of projections used to compute an image.

The source may be a fan beam directed toward a linear array of detectors, or a cone beam directed towards a two-dimensional detector array. Often, a dedicated investigative CT scanner will use a fan beam illuminating a linear or a narrow array in order to yield a high-quality image. Such scanners often rotate at a high speed around the patient (or object) under investigation in order to produce an image within a short period of time and to minimise movement artefacts in the image.

Other CT arrangements include a cone-beam arrangement mounted on or as part of the gantry of a radiotherapy apparatus, with the aim of combining radiotherapeutic treatment with obtaining a CT scan. The results of the CT scan can then confirm accurate positioning of the patient and/or guide the radiotherapy delivery. In such cases, the rotational speed of the CT scanner is often dictated by the rotational speed of the radiotherapy gantry, and may be as low as 1 rpm. Such combined systems may use a separate lower-energy diagnostic x-ray source for CT mounted (for example) 90° away from the therapeutic source, or may rely on a single source able to switch between a lower-energy diagnostic beam and a higher energy therapeutic beam. Sometimes, a form of CT (“portal CT”) is possible using images derived from the therapeutic beam after attenuation by the patient, but the attenuation coefficients of different materials become more similar at higher beam energies, so better contrast in the image is available at lower beam energies.

The therapeutic beam of a radiotherapy apparatus is collimated so as to limit its extent and confine the irradiation to those areas of the patient where it is required. This, together with rotation of the source around the patient enables the dose distribution to be closely controlled so that a high dose is applied to the site of the tumour (or other lesion), a relatively low dose is applied to the surrounding tissue, and (potentially) substantially no dose is delivered to sensitive structures such as the spinal cord. Those collimators are fixed to the radiation head from which the beam emanates and are moveable into and out of the beam so as to limit its overall extent. Typically, they include one or more block collimators, and/or “multi-leaf collimators” or MLCs, which consist of an array of adjacent thin leaves that can each extend into and out of the beam individually so as to shape the beam to a desired shape. An example of an MLC is shown in EP-A-0,314,214.

### SUMMARY OF THE INVENTION

Combined systems that rely on a single source able to switch between a diagnostic beam and a therapeutic beam have the advantage that they provide a true “beam's eye view” for the CT scanner, in that the scan is correlated exactly with the therapeutic beam as it is derived from beams emitted by the same source or, at least, emitted along the same axis. However, they suffer from the potential drawback that the beam must pass through the same collimation apparatus as the therapeutic beam. Whilst the collimators can be opened to their maximum aperture, this is often not as wide as would be the case if they were entirely absent. This limits the aperture of the projections from which the CT scan is derived and therefore limits the size of the CT reconstruction that can be obtained.

The present invention therefore provides a radiotherapeutic apparatus comprising a rotatable mount, a source of penetrating radiation mounted on the rotatable mount and able to emit, selectably, a first beam or a second beam along a beam axis, the first beam having an energy greater than the second beam, and at least one collimator located around the beam axis thereby to selectably limit a lateral extent of the beam and being rotateable around an axis parallel to the beam axis, and a control unit arranged, when the source emits the second beam, to rotate the collimator into a predetermined rotational position.

The penetrating radiation is typically x-radiation, but may be an alternative such as an electron beam.

The first beam and second beams are usually a therapeutic and a diagnostic beam, respectively. Typically, a useful therapeutic beam has a photon energy of between about 5 and 15 MeV. A diagnostic beam usually has a photon energy which is less than this, ideally below about 3 MeV. Separate sources which produce a dedicated diagnostic beam typically produce a beam at around 100-120 keV whereas combined accelerators able to switch between therapeutic and diagnostic beams have a diagnostic beam of around 1 to 3 MeV, typically about 1.4 MeV.

The collimator is preferably a multi-leaf collimator. Ideally, the collimator (of whatever sort) is opened by the control unit to its maximum extent when the source is emitting the second beam.

The predetermined position is, for a collimator with a rectangular aperture (often a square), one in which a diagonal of the aperture lies transverse to the plane swept out by the beam axis during rotation of the mount. More generally, where the aperture has at least one straight edge, the predetermined position is one in which the straight edge lies at an oblique angle to the plane swept out by the beam axis during rotation of the mount. This allows the aperture to have the maximum “reach” away from the beam axis, thus expanding the (three-dimensional) aperture of the CT system.

The axis of rotation of the collimator can be co-incident with the beam axis, as collimators are sometimes rotated during treatment in order to better match the shape created by the collimator to the shape of the tumour or other lesion being treated.

Alternatively, the axis of rotation of the collimator may be positioned so as to suit the needs of the CT imaging system.

The axis of rotation of the mount will usually intersect with the beam axis, to define an “isocentre” at the point of intersection which lies in the centre of the beam at all rotational positions of the mount.

The apparatus will usually include a detector for at least the second beam, mounted on the rotatable mount opposite the source in order to capture the projection images from the CT volume image is created. The detector may be a planar or flat-panel detector. It is preferably offsettable such that the beam axis meets the detector at a point non-coincident with the centre of the detector; this allows the detector to make best advantage of the additional “reach” created by rotating the collimator.

### BRIEF DESCRIPTION OF THE DRAWINGS

An embodiment of the present invention will now be described by way of example, with reference to the accompanying figures in which;

FIG. 1 shows a schematic vertical section through a radiotherapy apparatus

FIG. 2 shows a view along the rotation axis of the mount of a typical radiotherapy apparatus;

FIG. 3 illustrates the advantage of offsetting the detector;

FIG. 4 shows the view along the beam axis of a radiotherapy apparatus;

FIG. 5 shows the advantage relative to FIG. 4 of offsetting the detector; and

FIG. 6 shows the advantage relative to FIG. 5 of rotating the collimator.

### DETAILED DESCRIPTION OF THE EMBODIMENTS

Referring to FIG. 1, this shows in schematic form the general process by which a therapeutic beam of radiation is produced in a typical linear-accelerator based radiotherapy apparatus. A beam of high-energy electrons 10 is produced by a linear accelerator (not shown) and is directed towards an x-ray target 12 on which it impinges, producing x-rays 14. These are limited to a generally cone-shaped beam 16 by a primary collimator 18, which consists of a substantial block of metal with a cone-shaped through-aperture. X-rays passing through the aperture are allowed to continue, whereas those which impinge on the block are absorbed.

The beam 16 is then further collimated and shaped by a block collimator 20 and an MLC 22. The block collimator 20 consists of a pair of substantial blocks 24, 26 of a suitable radiopaque material such as tungsten, which can be moved in and out of the beam (in the x direction) from either side. Each block has a generally flat front edge which extends across the entire aperture of the beam and which may be rounded in the z direction (i.e. along the beam axis 28) in order to reduce penumbra.

The MLC 22 comprises a pair of frames 30 placed either side of the beam and spaced apart in the y direction, one of which is shown in FIG. 1. Each frame contains an array of thin leaves 32, arranged side-by side in the x direction. Each leaf is movable independently under the control of drive motors (not shown) which drive the leaves longitudinally (i.e. in the y direction) such that they can project out of the frame, into the beam aperture. The tips of the leaves thus define an undulating edge whose shape can be controlled as desired. Together, the collimators allow close control of the lateral extent of the beam and permit complex dose distributions to be delivered.

As shown in FIG. 1, the MLC 22 precedes the block collimator 20 along the beam path 28. This need not be the case, however, and the design of the radiotherapeutic apparatus can be adjusted as necessary. Additionally, in practice the beam generation part of the apparatus (shown in FIG. 1) will rotate around the patient as described below and may therefore adopt any orientation. FIG. 1 also shows only the relevant parts of the radiotherapeutic apparatus, with many other parts being present in practice.

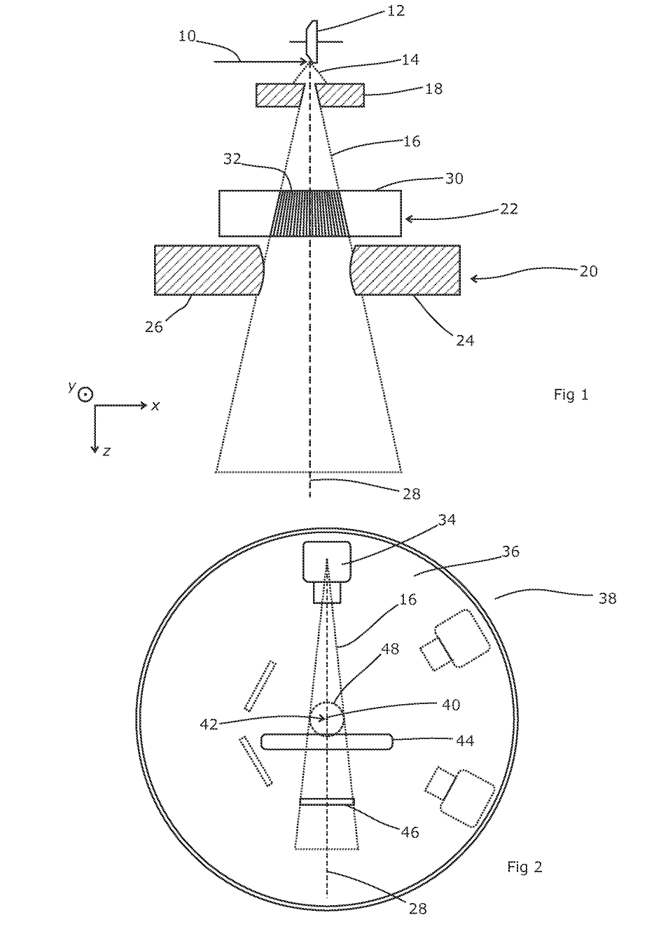

FIG. 2 shows the larger radiotherapy apparatus, including the radiation head 34 within which the structures shown in FIG. 1 are contained. This is mounted on a gantry arm (not visible) which projects from a mount 36 which is usually integrated into a suitably convenient wall 38 which may have been constructed for the purpose. The mount 36 is rotatable around an axis 40, with the gantry arm being fixed to the mount 36 at a point spaced from the axis 40. The radiation head 34 is oriented so that the beam 16 is directed towards the rotation axis 40, with the central axis 28 of the beam passing through the axis 40. This means that as the mount 36 rotates, taking the radiation head 34 with it (as shown dotted), the beam 16 always passes through the “isocentre” 42 at which the beam axis 28 and the rotation axis 40 meet, but does so from all possible directions. In combination with careful control of the collimators 20, 22, this allows a dose to be built up with minimal dose being delivered to surrounding tissue.

A patient table 44 is provided just below the isocentre 42, and can support a patient so that their tumour or other lesion is at or near the isocentre 42. Generally, such tables 44 are adjustable in all six degrees of freedom so as to allow the position of the patient to be closely adjusted to conform to that needed or expected by the radiotherapeutic process.

The radiation head 34 is controllable to produce x-rays of one of a number of different photon energies. In this case, the head 34 can produce one of two beams, a 1.4 MeV diagnostic beam suitable for preparing CT images and a high-energy therapeutic beam in the 5-15 MV range. A flat-panel detector 46 is attached to the mount 36, opposite the radiation head so that it lies in the path of the beam 16 with the patient table 44 between the detector 46 and the radiation head 34. The detector 46 is suited to the diagnostic beam and can therefore capture a projection image of a patient on the patient table 44; as the mount rotates, many such images can be captured allowing a CT image of the patient to be reconstructed. This allows a CT reconstruction to be prepared which is exactly correlated to the view of the therapeutic beam, as it is reconstructed from images obtained via the same source.

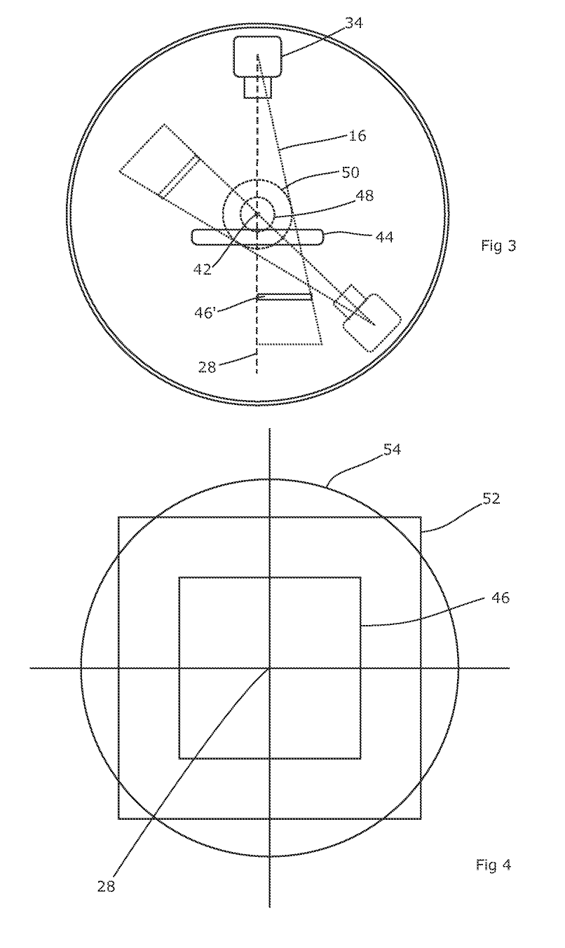

For a given size of detector 46 placed symmetrically under the beam 16, the maximum volume which can be imaged in this way is a cylinder around the isocentre shown by the dotted circle 48 in FIG. 2. This cylinder is the volume around the isocentre 42 that remains in the imaged part of the beam 16 as the radiation head 34 rotates around the isocentre 42. This volume can be increased by offsetting the detector as shown in FIG. 3, where the radiation head 34 emits a beam 16 towards a detector 46′ which is offset relative to the centreline 28 of the radiation head 34. The beam 16 can be shaped into an offset shape by use of the collimators 20, 22, if required. In this way, the imaged volume 50 around the isocentre 42 that is swept out as the radiation head 34 rotates (shown dotted) around the isocentre 42 is nearly doubled relative to the imaged volume 48 of FIG. 2. Note, however, that at any one moment only half of the imaged volume 50 is captured in a projection image but that the other half of the volume will be captured after the radiation head has rotated through 180°. The volume is usually not quite doubled in this way as it is advisable to allow a small overlap around the isocentre so that the projection images that are 180° apart can be matched without leaving an un-imaged gap between them. Thus, a larger part of a patient on the support 44 (or a larger patient) can be imaged.

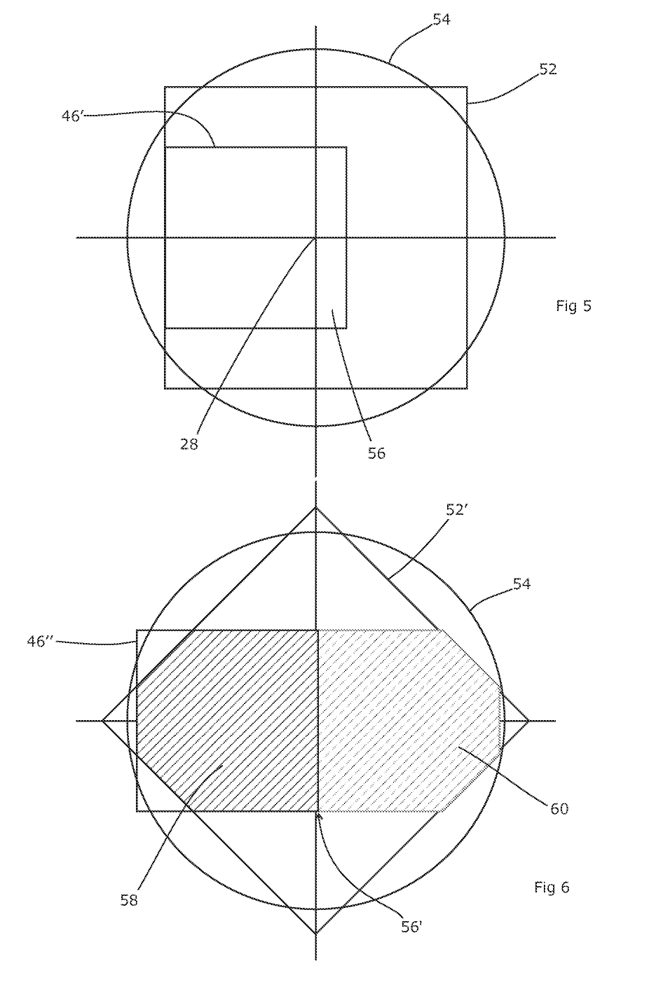

FIGS. 4 and 5 show this from a view along the beam axis 28 of the radiation head 34. In FIG. 4, a centrally mounted detector 46 does not make use of the beam aperture 52 made possible by fully-withdrawn collimators; this could be remedied by use of a larger detector, but there are technical difficulties in reliably extending the size of a flat-panel detector. Instead, as shown in FIG. 5, the detector 46′ can be offset relative to the beam centreline 28 and thereby take up the full extent of the aperture available through the withdrawn collimators 52. This means that the area swept out as the radiation head rotates corresponds to the area 46′ together with is reflection in the vertical axis (of FIG. 5), imaged by the detector panel after a 180° rotation.

A healthy overlap 56 is allowed for, as there is no benefit in offsetting the detector panel 46′ so far that it extends beyond the maximum collimator aperture 52. Generally, the dimensions of panels and collimators that are in current use mean that the collimator aperture 52 is the limiting factor. Also, the maximum usable beam aperture 54 permitted by the primary collimator 18 is wider than the collimator aperture 52, so some of the beam is wasted. FIG. 6 shows how this can be used according to the present invention. By rotating the square collimator aperture 52′ through 45°, the detector 46″ can be offset further, along the (now) diagonal of the collimator aperture. This moves the detector 46″ further towards the limit 54 imposed by the primary collimator 18 and by the need for a small overlap 56′ at the isocentre 28. The total area swept out is the combined areas 58 and 60, somewhat larger than the combined areas 46′ and 54.

This solution is shown for a square collimator aperture 52, but if this is a different shape then the angle of rotation may need to be adjusted accordingly. For example, if the collimator aperture is a non-square rectangle then the angle of rotation can be whatever is needed in order to place a diagonal of the rectangle substantially along the direction of rotation. Other shapes of collimator could be accommodated in like manner and as defined above.

The detector is shown in an offset manner partly for explanatory reasons and partly because current designs of detector have dimensions that call for this approach to be adopted. However, in some designs of radiotherapy apparatus, the collimator aperture may be small enough or the detector large enough that an offset for the detector is not needed.

It will of course be understood that many variations may be made to the above-described embodiment without departing from the scope of the present invention.

1. Radiotherapeutic apparatus comprising:
a rotatable mount,
a source of penetrating radiation, mounted on the rotatable mount, and able to emit, selectably, a first beam or a second beam along a beam axis, the first beam having an energy greater than the second beam;
at least one collimator, located around the beam axis thereby to selectably limit a lateral extent of the beam, and being rotateable around an axis parallel to the beam axis;
a control unit arranged, when the source emits the second beam, to rotate the collimator to a predetermined position.

2. Radiotherapeutic apparatus according to claim 1 in which the penetrating radiation is x-ray.

3. Radiotherapeutic apparatus according to claim 1 in which the first beam has an energy of at least 5 MeV.

4. Radiotherapeutic apparatus according to claim 1 in which the second beam has an energy of less than 3 MeV.

5. Radiotherapeutic apparatus according to claim 1 in which the second beam has an energy of at least 100 keV

6. Radiotherapeutic apparatus according to claim 1 in which the second beam has an energy of at least 1 MeV.

7. Radiotherapeutic apparatus according to claim 1 in which the collimator is a multi-leaf collimator.

8. Radiotherapeutic apparatus according to claim 1 in which the control unit is arranged, when the source emits the second beam, to open the collimator to its maximum extent.

9. Radiotherapeutic apparatus according to claim 1 in which the collimator has an aperture with at least one straight edge, and the predetermined position is one in which the straight edge lies at an oblique angle to the plane swept out by the beam axis during rotation of the mount.

10. Radiotherapeutic apparatus according to claim 1 in which the collimator has a rectangular aperture, and the predetermined position is one in which a diagonal of the rectangular aperture lies transverse to the plane swept out by the beam axis during rotation of the mount.

11. Radiotherapeutic apparatus according to claim 1 in which the collimator has a square aperture.

12. Radiotherapeutic apparatus according to claim 1 in which the collimator has a square aperture, and the predetermined position is one in which an edge of the square aperture lies at an angle of 45° to the plane swept out by the beam axis during rotation of the mount.

13. Radiotherapeutic apparatus according to claim 1 in which the axis of rotation of the collimator is co-incident with the beam axis.

14. Radiotherapeutic apparatus according to claim 1 in which the axis of rotation of the mount intersects with the beam axis.

15. Radiotherapeutic apparatus according to claim 1 further comprising a detector for at least the second beam, mounted on the rotatable mount opposite the source.

16. Radiotherapeutic apparatus according to claim 15 in which the detector is a planar detector.

17. Radiotherapeutic apparatus according to claim 15 in which the detector is offsettable such that the beam axis meets the detector at a point non-coincident with the centre of the detector.
